(19) United States
(12) Patent Application Publication
(10) Pub. No.: US 2024/0408359 A1
(43) Pub. Date: Dec. 12, 2024

(54) SHUNTING SYSTEMS AND METHODS, INCLUDING SYSTEMS AND METHODS FOR DELIVERING AND DEPLOYING THE SAME

(71) Applicant: Shifamed Holdings, LLC, Campbell, CA (US)

(72) Inventors: Brian Fahey, Menlo Park, CA (US); Scott Robertson, San Jose, CA (US); Anthony Pantages, San Jose, CA (US)

(21) Appl. No.: 18/698,328

(22) PCT Filed: Oct. 13, 2022
(86) PCT No.: PCT/US2022/046584

§ 371 Date: Apr. 3, 2024

Related U.S. Application Data
(60) Provisional application No. 63/255,279, filed on Oct. 13, 2021.
(60) Provisional application No. 63/302,533, filed on Jan. 24, 2022.

(51) Int. Cl. A61M 27/00; A61M 25/00; A61M 39/10

(52) U.S. Cl. CPC A61M 27/002; A61M 25/0074; A61M 25/0082; A61M 2039/1022; A61M 2205/0266; A61M 2210/125

(57) ABSTRACT

The present technology provides shunting systems that include an anchoring feature, a first canister, a second canister, and a tethering element coupling the first canister to the second canister. The tethering element can (a) orient the first canister and the second canister in a predetermined configuration when the system is deployed from a catheter, and (b) retain the first canister and the second canister in the predetermined configuration following deployment of the system.

### CROSS REFERENCE TO RELATED APPLICATION(S)

This application claims the benefit of U.S. Provisional Patent Application No. 63/255,279, filed Oct. 13, 2021, and U.S. Provisional Patent Application No. 63/302,533, filed Jan. 24, 2022, both of which are incorporated by reference herein in their entireties.

### TECHNICAL FIELD

The present technology generally relates to implantable medical devices and, in particular, to implantable shunting systems and systems and methods for delivering and deploying the same.

### BACKGROUND

Shunting systems have been widely proposed for treating various disorders associated with fluid build-up or pressure in a particular body region. For example, interatrial shunting systems that shunt blood from the left atrium of the heart to the right atrium of the heart have been proposed as a treatment for heart failure in general, and heart failure with preserved ejection fraction in particular. Proposed shunting systems range in complexity from simple tube shunts to more sophisticated systems having on-board electronics, adjustable lumens, or the like. Despite the advancement of shunting system technology, designing shunting systems that can be reliably and relatively non-invasively delivered and deployed across a target structure remains a challenge.

### BRIEF DESCRIPTION OF THE DRAWINGS

FIGS. 1A-1C illustrate an adjustable shunting system in a deployed configuration and configured in accordance with select embodiments of the present technology.

FIGS. 2A-2G illustrate various stages of an operation of delivering the adjustable shunting system of FIGS. 1A-IC to a septal wall of a heart, and of deploying the system across the septal wall, in accordance with embodiments of the present technology.

FIGS. 3A-3C illustrate another adjustable shunting system in a deployed configuration and configured in accordance with select embodiments of the present technology.

FIG. 4 illustrates the adjustable shunting system of FIGS. 3A-3C positioned within a delivery device and configured in accordance with select embodiments of the present technology.

FIGS. 5A and 5B illustrate the adjustable shunting system of FIGS. 3A-3C deployed across the septal wall in accordance with embodiment of the present technology.

FIG. 6A illustrates an adjustable shunting system in a deployed configuration and configured in accordance with another embodiment of the present technology.

FIG. 6B illustrates an adjustable shunting system in a deployed configuration and configured in accordance with yet another embodiment of the present technology.

FIGS. 7A and 7B illustrate canisters for use with the adjustable shunting systems of the present technology and configured in accordance with select embodiments of the present technology.

### DETAILED DESCRIPTION

The present technology is directed to shunting systems for shunting fluid between a first body region and a second body region, and to systems and methods of delivering and deploying the same. In some embodiments, for example, the present technology includes a shunting system having an anchoring feature configured to extend across a tissue wall separating a first body region and a second body region and define an artificial opening therebetween. The system can further include a first canister coupled to the anchoring feature and positionable in the first body region and a second canister coupled to the anchoring feature and positionable in the second body region. The first and second canisters can house various electrical components of the system. The system can also include a mechanical connection, such as a tethering element, extending between the first and second canisters. The tethering element can (a) orient the first canister and the second canister in a predetermined configuration when the system is deployed from a catheter, and (b) retain the first canister and the second canister in the predetermined configuration following deployment of the system. For example, in some embodiments the tethering element can be composed at least partially of a material that exhibits elastic or superelastic properties at body temperature, such as Nitinol having an austenite finish temperature of less than body temperature. In some embodiments, the tethering element can include electrical lead wires that allow for signals to be sent between components in the first canister and components in the second canister. In some embodiments, one or more canisters can include more than one tethering element. In some embodiments, the tethering element may include a first loop portion adjacent the first canister and a second loop portion adjacent the second canister that further bias the first canister and the second canister toward the predetermined configuration.

Without being bound by theory, and as described in detail below, the systems and methods described herein are expected to improve both (a) the process of delivering shunting systems, and (b) the functioning of shunting systems once deployed. For example, the tethering elements described herein are expected to simplify the delivery process by assisting with positioning various components of the shunting systems during delivery. The tethering elements are also expected to help retain various components in a desired position following deployment. Other advantages of the present technology will become apparent based on the description below.

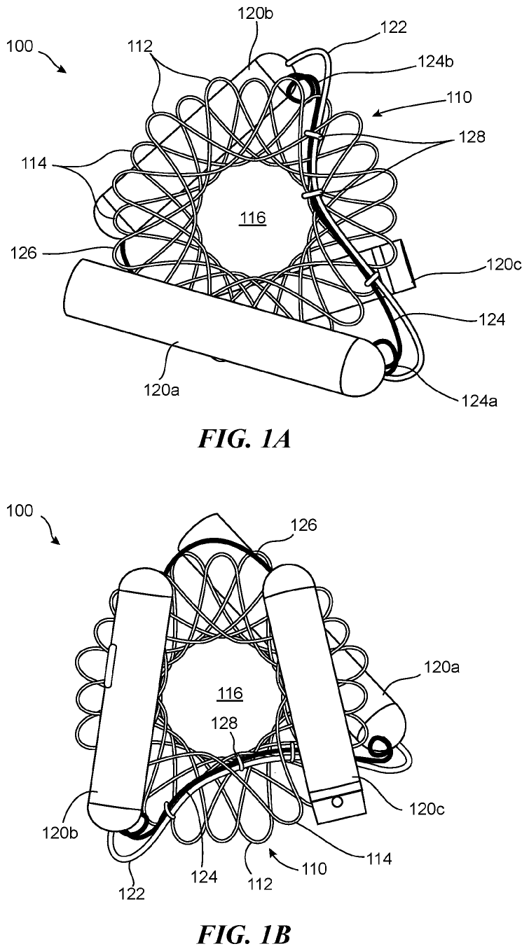

The terminology used in the description presented below is intended to be interpreted in its broadest reasonable manner, even though it is being used in conjunction with a detailed description of certain specific embodiments of the present technology. Certain terms may even be emphasized below: however, any terminology intended to be interpreted in any restricted manner will be overtly and specifically defined as such in this Detailed Description section. Additionally, the present technology can include other embodiments that are within the scope of the examples but are not described in detail with respect to FIGS. 1A-6B.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

FIGS. 1A-IC illustrate a shunting system 100 (“the system 100”) in a deployed configuration and configured in accordance with select embodiments of the present technology. More specifically, FIG. 1A is a front view of the system 100 in the deployed configuration, FIG. 1B is a view of the system 100 in the deployed configuration with the system 100 rotated 180 degrees relative to the view shown in FIG. 1A, and FIG. 1C is a side view of the system 100 in the deployed configuration. As described in detail below; the system 100 can be configured to shunt fluid between a first body region and a second body region when implanted in a patient. For example, the system 100 can be an interatrial shunting system configured to be implanted across a septal wall of a patient to shunt blood from the left atrium to the right atrium.

Referring collectively to FIGS. 1A-IC, the system 100 includes an anchoring or stabilizing feature 110 configured to secure the system 100 to patient tissue and/or stabilize the position of system 100 in a desired anatomic location. In the illustrated embodiment, the anchoring feature 110 is a wire or filament structure (e.g., a braided or woven wire structure) having a generally annular geometry and defining a central lumen 116 extending therethrough (which may also be referred to herein as an “opening.” or “passage”). The anchoring feature 110 further includes a first plurality of petals or appendages 112 and a second plurality of petals or appendages 114. In some embodiments, the wire forming pattern of the anchoring feature 110 results in immediately adjacent petals of the first petals 112 not being formed by an adjacent segment of the wire structure forming the anchoring feature 110. Instead, the wire structure can alternate between forming first petals 112 on a first side of the system 100 and second petals 114 on the second side of the system (e.g., the portion of the wire structure that forms an individual first petal 112 at the 12:00 position may traverse a central portion 115 of the anchoring feature 110 to form an individual second petal at the 3:00 position before traversing the central portion 115 to form another individual first petal 112, and so on and so forth) As best shown in FIG. 1C, the central portion 115 of the anchoring feature 110 that defines the lumen 116 separates the first plurality of petals 112 and the second plurality of petals 114 such that a gap 118 exists between the first plurality of petals 112 and the second plurality of petals 114. When the system 100 is deployed across a tissue structure (e.g., a septal wall), the gap 118 is configured to receive patient tissue. Additionally, the first plurality of petals 112 and the second plurality of petals 114 can be at least partially biased toward one another such that the first petals 112 and the second petals 114 at least partially squeeze patient tissue received within the gap 118 to secure the system 100 to patient tissue. For example, when deployed across the septal wall, the first petals 112 may reside within the left atrium, the second petals 114 may reside within the right atrium, and the gap 118 may receive a portion of the patient's septal wall (e.g., at the fossa ovalis). The first petals 112 may be biased at least slightly toward the second petals 114 (and/or the second petals 114 may be biased at least slightly toward the first petals 112) such that the anchoring feature 110 forms a slight clamping force on the portion of the septal wall within the gap 118. In some embodiments, the first petals 112 and the second petals 114 are at least partially staggered such that individual first petals 112 do not entirely overlap with individual second petals 114. In other embodiments, the first petals 112 and the second petals 114 may be biased away from other and/or have an outwardly tapered shape to accommodate an increasing thickness of the septal wall adjacent to an outer perimeter of the anchoring feature 110. Without being bound by theory, this is expected to spread the pinching force over a larger area of the septal wall.

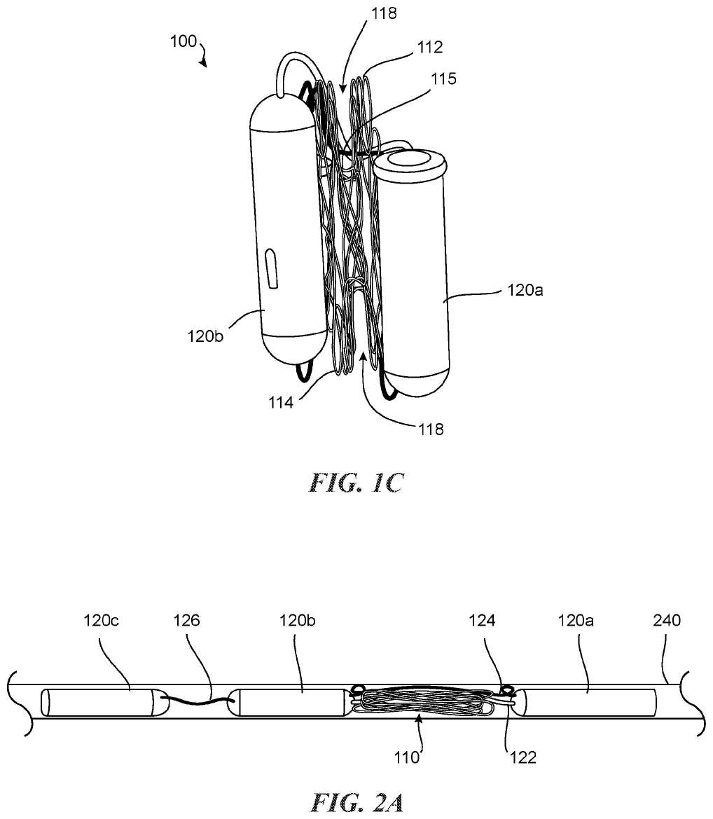

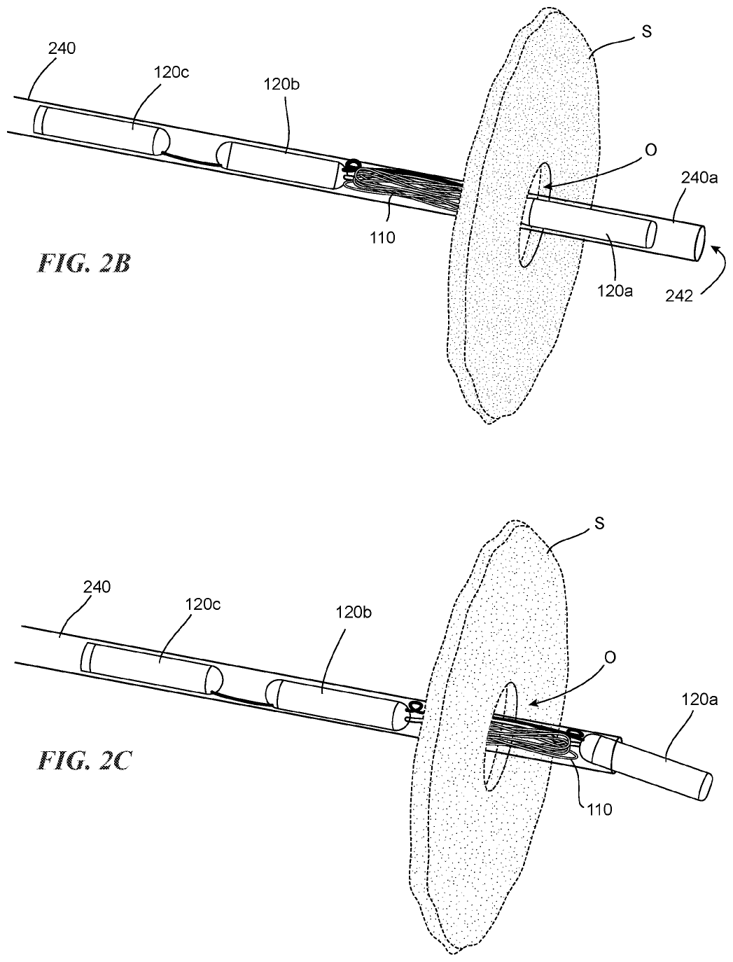

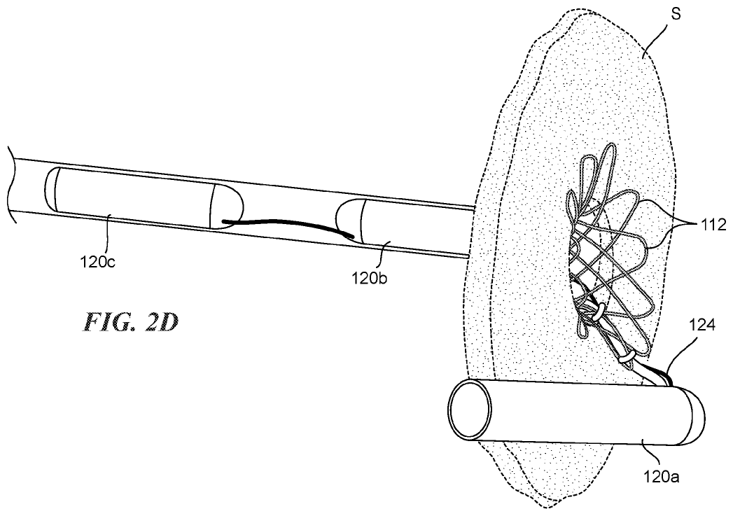

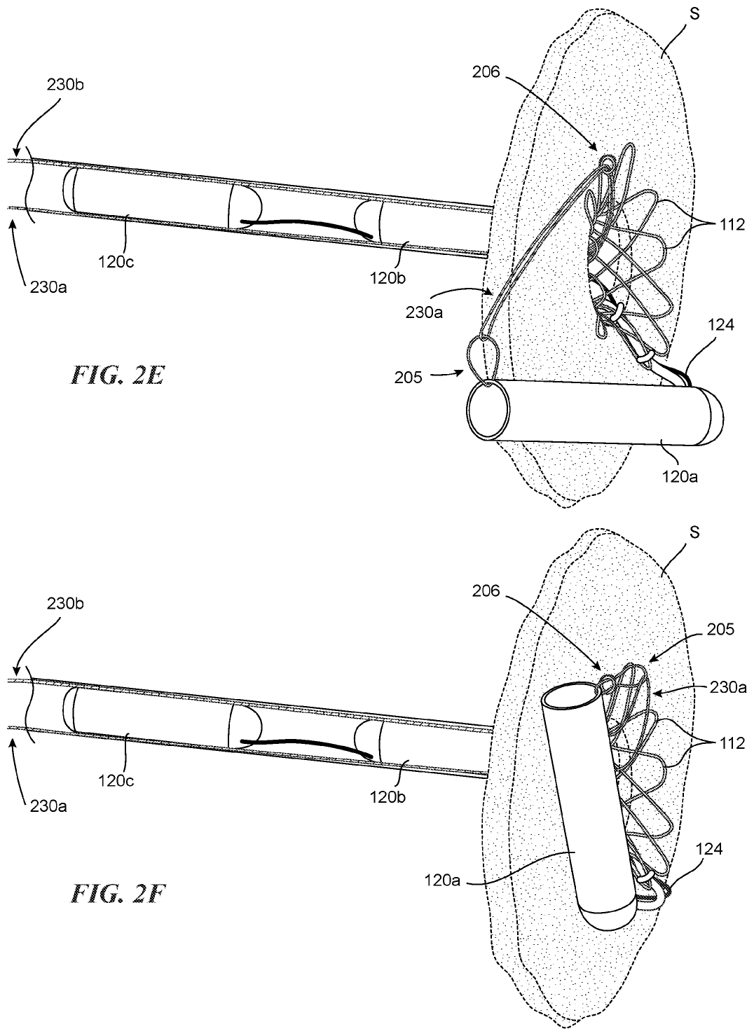

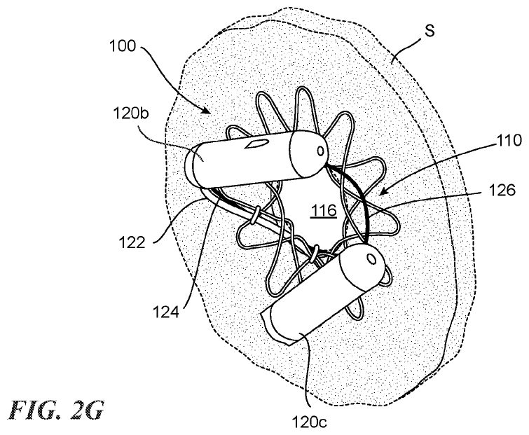

The anchoring feature 110 can be at least partially composed of a self-expanding material such that, after being exposed to stress and strain induced by being collapsed into a delivery tool (e.g., catheter, sheath, etc.) for delivery (e.g., as described with reference to FIGS. 2A-2G), it exhibits an elastic response to being deployed at body temperature. For example, the anchoring feature 110 can be composed of Nitinol that has an austenite finish temperature below body temperature. Accordingly, as described in detail with respect to FIGS. 2A-2G, the anchoring feature 110 can automatically deploy (e.g., self-expand without additional input or manipulation by a clinician) from a collapsed delivery configuration (e.g., as positioned in a delivery tool such as a catheter or sheath) to an expanded deployed configuration when released from the delivery tool. In some embodiments, the self-expanding or superelastic properties of the anchoring feature 110 may also enable the anchoring feature 110 to resist plastic mechanical deformation once deployed, and thus can provide a generally stable anchoring mechanism for the system 100. In other embodiments, the anchoring feature 110 can be composed of a material that is not self-expanding at body temperature. In one example, the anchoring feature 110 can be composed of Nitinol that has an austenite finish temperature above body temperature. In such an example, the anchoring feature 110 can be initially released from a delivery tool in a preliminary position (e.g., the collapsed delivery configuration, an intermediate configuration, etc.) and subsequently be heated above the austenite finish temperature to transition the shape of the anchoring feature 110 toward the deployed configuration. In a second example, the anchoring feature 110 can be composed of a material such as stainless steel (e.g., 316L), titanium alloy (e.g., TiAl₆V₄), cobalt chromium alloy (e.g., L605), or polymer (e.g., PEEK). Some implementations of the second example can be self-expanding based upon a geometric configuration of the feature 110. Other implementations can be manually expanded by an operator after an initial deployment using tools such as catheters, sutures, balloons, and the like.

In some embodiments, the anchoring feature 110 can be configured to receive and/or generate energy/power when communicatively coupled to an energy source (not shown) in addition to stabilizing the system 100 across the septal wall. For example, in some embodiments, the wire(s) or filament(s) utilized to form the first petals 112 and/or the second petals 114 constitute one or more inductive portions of a circuit configured to generate energy when exposed to an electromagnetic field. The energy generated by the anchoring feature 110 can be stored and/or used to power various active components of the system 100, as described in detail below. To facilitate inductive charging using the anchoring features 110, some or all of the anchoring feature 110 can include a relatively conductive material (e.g., silver). For example, the wire(s) or filament(s) that are used to construct the anchoring feature 110 can be highly conductive themselves (e.g., silver), have a highly conductive core surrounded by a less conductive (e.g., Nitinol) sheath or coating, a less conductive core with a highly conductive sheath or coating, a highly conductive wire coupled to a less conductive wire, or another suitable arrangement. In such embodiments involving a composite structure, the less conductive portion generally provides the majority of the structural performance (e.g., anchoring) whereas the more conductive portion generally provides the majority of the electrical performance (e.g., energy/power transfer).

The system 100 further includes a plurality of canisters 120 (e.g., shown in FIGS. 1A-IC as a first canister 120a, a second canister 120b, and a third canister 120c). The canisters 120 are specialized (e.g., hermetically sealed) containers that house various electronic components of the system 100. For example, the canisters 120 can house one or more energy storage components (e.g., a battery, a capacitor, a supercapacitor, etc.), one or more sensors or associated electronic circuitry (e.g., pressure sensors), an actuator (e.g., a motor), one or more data storage elements, one or more telemetry components, one or more microcontrollers, one or more processors, one or more radios, or the like.

In the illustrated embodiment, the first canister 120a resides on (e.g., is coupled to) or near a first side (e.g., left atrium) of the anchoring feature 110, and the second canister 120b and the third canister 120c reside on (e.g., are coupled to) a second side (e.g., right atrium). In other embodiments, however, the first side (e.g., left atrium) may include a plurality of canisters, and the second side (e.g., right atrium) can include a single canister. Of course, the system 100 may include more or fewer canisters, such as one, two, three, four, five, six, or more canisters. Moreover, any number of canisters (including zero) can reside on the first side (e.g., left atrium) of the anchoring feature 110, and any number of canisters (including zero) can reside on the second side (e.g., right atrium) of the anchoring feature 110.

In the deployed configuration, the canisters 120 can fully or at least partially overlap with the first petals 112 and/or the second petals 114, e.g., as opposed to be positioned radially outward from the tips of the first petals 112 and/or the second petals 114. Similarly, the canisters 120 can be positioned in apposition with the first petals 112 and/or the second petals 114. This is expected to reduce the overall footprint of the system 100, and advantageously avoid blocking, contacting, or otherwise interfering with other anatomical structures that may be adjacent the system 100 (e.g., the mitral valve, the tricuspid valve, the coronary sinus, etc.). As described below, the positioning of the canisters 120 can be determined via one or more mechanical connections that bias the canisters 120 toward a desired position.

The canisters 120 can be mechanically and/or electrically coupled. For example, the first canister 120a can be mechanically coupled to the second canister 120b via a first tethering element 124, and the second canister 120b can be mechanically coupled to the third canister 120c via a second tethering element 126. Because the first canister 120a and the second canister 120b reside on opposite sides of the anchoring feature 110, the first tethering element 124 traverses the anchoring feature 110. For example, the first tethering element 124 can extend through the lumen 116 formed by the anchoring feature 110. In some embodiments, the first tethering element 124 is interlaced with (e.g., woven through) one or more first petals 112 and/or second petals 114 as it extends across the anchoring feature 110.

The first and second tethering elements 124, 126 can orient the canisters 120 in a desired position relative to the anchoring feature 110 and relative to one another. For example, in some embodiments the first and second tethering elements 124, 126 can be composed of shape-memory material (e.g., shape memory alloys such as Nitinol, NiTiCu, NiTiPd, AgCd, AuCd, etc., or shape memory polymers such as bio-polyethylene terephthalate) that biases the canisters 120 toward a desired position (e.g., the first and second tethering elements 124, 126 bias the canisters 120 toward the positions shown in FIGS. 1A-IC). As another example, the first and second tethering elements 124, 126 can be composed of an elastic material that likewise biases or holds the canisters 120 toward a desired position. As yet another example, the first and second tethering elements 124, 126 can be composed of a rigid (or at least semi-rigid) and inflexible material, such as stainless steel. In yet another example, the tethering elements 124, 126 can be one or more sutures. Accordingly, the first and second tethering elements 124, 126 can at least partially hold the canisters 120 in the desired position even if deformation forces are applied against the canisters 120. As described below with respect to FIGS. 2A-2G, this can also help assist with deploying the canisters 120 into the desired position when the system 100 is deployed from a catheter.

In some embodiments, the first tethering element 124 can include a first loop portion 124a proximate the first canister 120a and a second loop portion 124b proximate the second canister 120b. In some embodiments, the first and second loop portions 124a,b act as torsion spring-like elements that follow conventional spring design principals in which the force generated by the loops is proportional to the spring stiffness and the displacement of uncoiling. Without being bound by theory, the loop portions 124a, 124b can increase the stored mechanical energy within the tethering element 124, and therefore are expected to increase the biasing forces generated by the tethering element 124, thereby promoting self-expansion and stabilization of the tethering element 124. This is expected to further assist in (a) deploying the canisters 120 to the desired position during deployment, and (b) holding the canisters 120 at the desired position once deployed. Without being bound by theory, the loops portions 124a, 124b can also decrease the material strains when the system 100 is collapsed into a catheter. This is expected to provide self-expansion of the tethering element 124 when it is constructed using conventional linear-elastic materials (e.g., stainless steel, titanium alloys, cobalt-chromium alloys) using spring design knowledge. In some embodiments, however, one or both of the loop portions 124a, 124b are omitted, and the tethering element 124 is substantially linear (e.g., similar to the second tethering element 126).

In some embodiments, the first and second tethering elements 124, 126 are composed at least partially of Nitinol. In such embodiments, the first and second tethering elements 124, 126 generally have an austenite finish temperature less than body temperature so that the first and second tethering elements 124, 126 exhibit superelastic properties at body temperature. However, in other embodiments the first and second tethering elements 124, 126 can have an austenite finish temperature above body temperature (e.g., greater than 40 degrees Celsius, greater than 45 degrees Celsius, etc.), such that the first and second tethering elements 124, 126 can be selectively transitioned between a first deformable or plastic material state (e.g., when at body temperature) and a second superelastic material state (e.g., when heated above the austenite finish temperature).

The canisters 120 may also be connected via an electrical connection. In the illustrated embodiment, for example, the first canister 120a is electrically coupled to the second canister 120b via an electrical connection 122. Likewise, although not illustrated, the third canister 120c can be electrically coupled to one or both of the first canister 120a or the second canister 120b via an electrical connection. The electrical connection 122 is configured to transmit electrical signals (e.g., energy, data, etc.) between the first canister 120a and the second canister 120b. For example, the electrical connection 122 can be a conductive wire or filament. Accordingly, energy stored in one canister (e.g., the second canister 120b) can be used to power active components stored in another canister (e.g., the first canister 120a) by virtue of the electrical connection 122. By further example, a microcontroller in one canister (e.g., the first canister 120a) can send instructions via electrical connection 122 to control the operation of a sensor in a second canister (e.g., the second canister 120b), and through the same or an additional electrical connection 122 the data from the sensor can be transferred and stored in memory located in a third canister (e.g., the third canister 120c).

In some embodiments, the electrical connection 122 can extend alongside the first tethering element 124. However, in many embodiments, the electrical connection 122 and the first tethering element 124 are generally distinct components. This is because the first tethering element 124 can experience relatively high levels of internal stress and strain at the connection points to the first canister 120a and the second canister 120b (e.g., due to the tight bend radius at the first loop portion 124a and the second loop portion 124b, respectively). Such high levels of stress and strain may not be suitable if applied to the conductive wire of the electrical connection 122. Accordingly, decoupling the electrical connection 122 from the first tethering element 124 can allow the tethering element 124 to manipulate the first canister 120a in a desired way for delivery while not applying excessive forces to the electrical connection 122. In some embodiments, the electrical connection 122 and the first tethering element 124 can be coupled together at intermediate portions via one or more sutures 128. However, such arrangement is not expected to induce excessive stress or strain in the electrical connection 122 because the end portions of the electrical connection 122 and the first tethering element 124 remain decoupled. In yet other embodiments, the electrical connection 122 and the first tethering element 124 are a single element that can optionally include other features for mitigating the effects of stress or strain on the electrical connection portion.

FIGS. 2A-2G illustrate various stages of an operation of delivering the system 100 across a septal wall of the patient in accordance with select embodiments of the present technology. Although FIGS. 2A-2G are described in the context of deploying the system 100 across the septal wall, one skilled in the art will appreciate that the same or similar procedure could be used to deploy the system 100 across other tissue walls dividing a first body region and a second body region.

FIG. 2A illustrates the system 100 positioned within a catheter 240 in a “delivery configuration” and configured in accordance with select embodiments of the present technology. In particular, the components of the system 100 are arranged in an “end-to-end” arrangement when loaded in the catheter 240. For example, the anchoring feature 110 does not overlap with any of the canisters 120 in the delivery configuration. This is expected to minimize the outer diameter of the system 100 in the delivery configuration, which in turn is expected to reduce the size of catheter needed to deliver the system 100, and thus reduce the invasiveness of the delivery procedure. For example, the system 100 can be collapsed such that it can be delivered in a 22 French catheter or smaller, a 24 French catheter or smaller, a 26 French catheter or smaller, a 28 French catheter or smaller, a 30 French catheter or smaller, etc. As a result, the system 100 may have an axial end-to-end length of between about 20 mm and 80 mm in the delivery configuration, compared with an axial end-to-end length of between about 5 mm and about 20 mm once deployed.

FIG. 2B shows the catheter 240 carrying the system 100 being advanced through an opening O in a septal wall S. In general, the catheter 240 is advanced from the right atrium and into the left atrium in accordance with known procedures for delivering transeptal and/or other cardiac implants. The opening O can be made by the catheter 240 as it is advanced from the right atrium to the left atrium, via a separate puncturing tool (not shown), or via other mechanisms (e.g., the opening can exist due to a previous procedure or as a result of a congenital defect). As illustrated, the catheter 240) is advanced until a distal end portion 240a of the catheter 240 having an opening 242 is positioned within the left atrium.

Referring next to FIG. 2C, once the distal end portion 240a of the catheter is in the left atrium, the system 100 can be advanced out of the opening 242 into the left atrium as the catheter is held stable or retracted toward the right atrium. As illustrated in FIG. 2C, this causes the first canister 120a to be pushed out the opening 242 and into the left atrium. As the catheter 240) is further retracted toward the right atrium and/or the system 100 is pushed distally, the anchoring feature 110 is also deployed out the opening 242 of the catheter 240, as shown in FIG. 2D. Because the anchoring feature 110 may exhibit self-expanding properties at body temperature as previously described, embodiments of the anchoring feature 110 can automatically expand outwardly toward its deployed configuration shown in FIGS. 1A-IC as it exits the catheter 240 without additional input or manipulation by a clinician. Accordingly, the anchoring feature 110 automatically expands radially outward to interface with the septal wall. In variation embodiments, the anchoring feature can either partially self-expand, or not self-expand upon deployment, and accordingly an additional operation (e.g., using balloons, sutures, pull-wires, etc.) must be undertaken to finalize the geometry of the anchoring feature into its desired deployment configuration. When fully deployed from the catheter 240, the anchoring feature 110 can contact both the left atrial side and the right atrial side of the septum as the tissue is held between the first petals 112 and the second petals 114. The catheter 240 can be further retracted until the second and third canisters 120b,c are also deployed. FIG. 2G illustrates the system 100 as viewed from the right atrium side following deployment.

The first and second tethering elements 124, 126 can help control movement of the canisters 120 during the delivery procedure. For example, once a canister (e.g., the first canister 120a) is deployed from the catheter 240, the first tethering element 124 rotates and/or transitions the canister into the desired orientation relative to the anchoring feature 110. This is because the external forces deforming the first tethering element and/or the canister(s) (i.e., the force exerted by the catheter 240) are removed, permitting the first tethering element 124 to move toward its default geometry and exert the forces required to shift the canisters into position. Likewise, once the third canister 120c is deployed from the catheter 240, the second tethering element 126 rotates and/or transitions one or more canisters (e.g., the second canister 120b and/or the third canister 120c) into the desired orientation relative to the anchoring feature 110. Moreover, the first and second tethering elements 124, 126 can be configured such that the canisters 120 remain proximate the anchoring feature 110 as they rotate or translate into the desired position. In other words, the first and second tethering elements 124, 126 can prevent the canisters 120 from migrating into other anatomical structures (e.g., the mitral valve) during the delivery process, in addition to assisting with correctly positioning the canisters.

In some embodiments, the first and second tethering elements 124, 126 deploy the canisters 120 into predetermined orientations that cannot be unintentionally manipulated following delivery. This is expected to be advantageous because it reduces the likelihood of the canisters 120 accidently being pushed out of position during or after delivery. In some embodiments, the first and second tethering elements 124, 126 may include or be coupled to additional features (e.g., sutures, locking mechanisms, etc.) that stabilize the canisters but allow for repositioning of the canisters 120 following deployment (e.g., via a physician).

In some embodiments, additional components can be utilized to assist with deploying and positioning the system 100 in a target orientation. For example, the canisters 120 can optionally have one or more features that lock into place during deployment to assist with retaining the canisters 120 in a desired orientation following deployment. For example, as shown in FIG. 2E, the system 100 can optionally include a first (e.g., male) mating feature 205 coupled to the first canister 120a and a second (e.g., female) mating feature 206 coupled to one of the petals 112. The first mating feature 205 can be mated with or otherwise locked to the second mating feature 206 to retain the first canister 120a in a desired orientation or position. For example, in the illustrated embodiment, the first mating feature 205 includes a loop-like flexible shape that can be flexed/straightened and pulled through the second mating feature 206. Once inserted through the second mating feature 206, the first mating feature 205 can return to its loop shape, thereby “locking” the first mating feature 205 to the second mating feature 206 (as shown in FIG. 2F).

To facilitate engagement of the mating features, in some embodiments, the delivery catheter 240) and/or the system 100 can include temporary tethers 230 (shown as a first temporary tether 230a and a second temporary tether 230b in FIG. 2E) that are coupled to one or more of the canisters 120 or other aspects of the system 100 and extend through the catheter 240) such that they are accessible external to the patient. In the illustrated embodiment, for example, the first temporary tether 230a is routed through both the first mating feature 205 and the second mating feature 206. The first temporary tether 230a can therefore be used to apply tension (e.g., by a user pulling on the first temporary tether 230a) to the first mating element 205 to both straighten it and pull it through the second mating element 206. When the tension is removed from the first temporary tether 230a, the first mating feature 205 can (automatically) return to its loop shape, thereby “locking” the first mating feature 205 to the second mating feature 206. Accordingly, the first temporary tether 230a, the first mating feature 205, and the second mating feature 206 can be used to further assist with positioning the first canister 120a in a desired position during deployment and/or retaining the first canister 120a in the desired position following deployment. Once the first mating feature 205 is locked to the second mating feature 206, the first temporary tether 230a can be removed from the catheter 240 and the patient. In some embodiments, the temporary tethers 230) are not removed following deployment of the system 100. In such embodiments, the temporary tethers 230 may optionally be composed of a bioabsorbable material such that the temporary tethers 230 will be adsorbed by the patient's body over time.

The system 100 can have similar mating features (not shown) on the left atrial side of the system 100 that can be connected (e.g., mated) by the second temporary tether 230b during a deployment and positioning procedure, e.g., to assist with positioning and/or retaining the second canister 120b and/or the third canister 120c in a desired position. Those skilled in the art will also recognize that the mating features can have designs and configurations other than those shown in FIG. 2E. For example, the second mating feature 206 can be designed to be flexible instead of the first mating feature 205, either the canister or the appendages may house the “male” or “female” mating features, and the shape of the mating features can be any number of configurations that can create a mechanical interlock (e.g., button and slit, arrow and hole, etc.). In some embodiments, the mating features can be configured to automatically engage as a result of biasing forces provided by the tethering elements 124, 126, e.g., in lieu of using the temporary tethering elements 230 to engage the mating features.

In some embodiments, the temporary tethers 230 can also perform other functions. For example, in some embodiments, the temporary tether(s) 230 can also assist with positioning the system 100 in the catheter 240. For example, tension may be applied to the temporary tether(s) 230 to pull the system 100 into the catheter 240. During deployment of the system 100 from the catheter 230, the self-expansion of the anchoring feature 110 can also be controlled by keeping tension on the temporary tether(s) 230. As the tension is lowered, the self-expanding anchoring feature 110 will slowly move towards its desired expanded geometry, thereby allowing more precise positioning and/or ability for repositioning of the anchoring system 110.

Once deployed across the septal wall S as shown in FIG. 2G, the system 100 can shunt blood between the left atrium and the right atrium. For example, blood can flow through the central lumen 116 extending through the central portion 115 (FIG. 1C) of the anchoring feature 110. In some embodiments, the system 100 may include additional features defining the lumen 116. For example, the system 100 can include one or more membranes (not shown) coupled to the central portion 115 (FIG. 1C). The system 100 may further yet include additional features not described with respect to FIGS. 1A-2E, such as one or more actuation mechanisms for adjusting a size, shape, geometry, and/or resistance of a flow path through the system 100. For example, the system 100 may include certain features described in U.S. Patent App. Publication No. 2021/0085935 and International Patent Publication No. WO 2021/113670, the disclosures of which are incorporated by reference herein in their entireties.

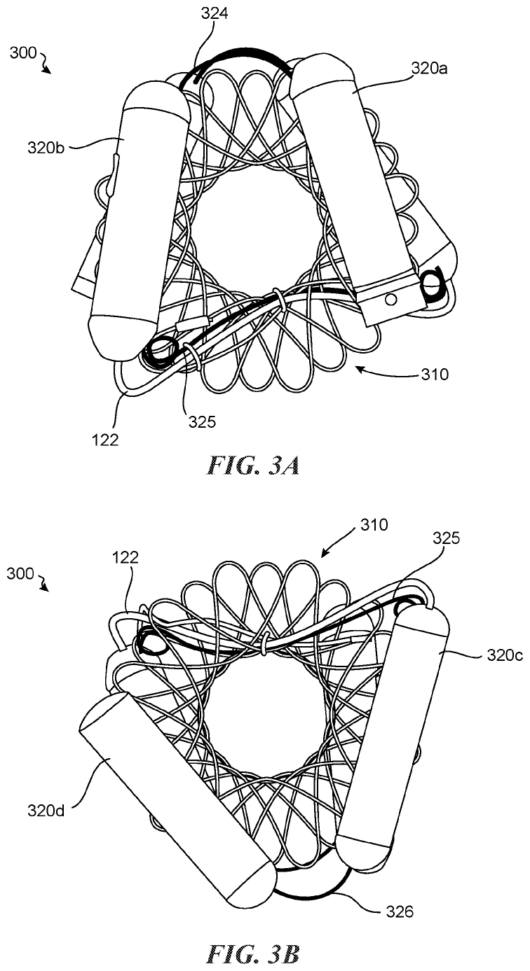

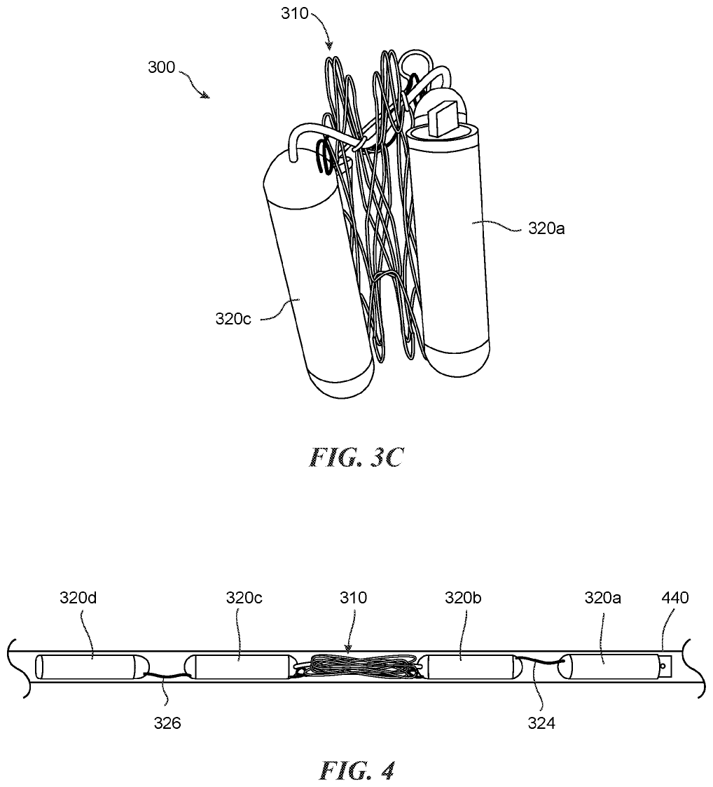

FIGS. 3A-3C illustrate another shunting system 300 configured in accordance with embodiments of the present technology. More specifically. FIG. 3A is a front view of the system 300 in the deployed configuration, FIG. 3B is another view of the system 300 in the deployed configuration with the system 300 rotated 180 degrees relative to the view shown in FIG. 3A, and FIG. 3C is a side view of the system 300 in the deployed configuration. Similar to the system 100 described with respect to FIGS. 1A-2E, the system 300 can be configured to shunt fluid between a first body region and a second body region when implanted in a patient. For example, the system 300 can be an interatrial shunting system configured to be implanted across a septal wall of a patient to shunt blood between the left atrium and the right atrium.

The system 300 can include certain features generally similar to the system 100 described with respect to FIGS. 1A-2G. For example, the system 300 can include an anchoring or stabilizing feature 310 that is the same as or generally similar to the anchoring feature 110 of the system 100. Relative the system 100, however, the system 300 includes four canisters 320) (shown as a first canister 320a, a second canister 320b, a third canister 320c, and a fourth canister 320d). In a deployed configuration, the first canister 320a and the second canister 320b reside on (e.g., are coupled to) or near a first portion (e.g., the portion intended to reside in the left atrium) of the anchoring feature 310, and the third canister 320c and the fourth canister 320d reside on (e.g., are coupled to) a second portion (e.g., the portion intended to reside in the right atrium) that can be generally opposite the first portion. The first canister 320a is mechanically coupled to the second canister 320b via a first tethering element 324, the second canister 320b is mechanically coupled to the third canister 320c via a second tethering element 325, and the third canister is mechanically coupled to the fourth canister 320d via a third tethering element 326. The first tethering element 324, the second tethering element 325, and the third tethering element 326 can be generally similar to the first tethering element 124 and/or the second tethering element 126 of the system 100, and thus can assist in deploying and positioning the canisters 320 relative to the anchoring feature 310 and/or relative to one another. The canisters 320 can also be electrically coupled via one or more electrical connections 322, which can be the same as or generally similar to the electrical connection 122 of the system 100. Although only the second canister 320b and the third canister 320c are shown as electrically coupled in FIGS. 3A-3C, in other embodiments any combination of the canisters 320 can be electrically coupled to one another and/or all canisters can be electrically coupled together.

FIG. 4 illustrates the system 300 positioned within a catheter 440 in a “delivery configuration” and configured in accordance with select embodiments of the present technology. In particular, and as described above for the system 100 with respect to FIG. 2A, the components of the system 300 are arranged in an “end-to-end” arrangement when loaded in the catheter 440). As previously described, this is expected to reduce the size of the catheter needed to deliver the system 300.

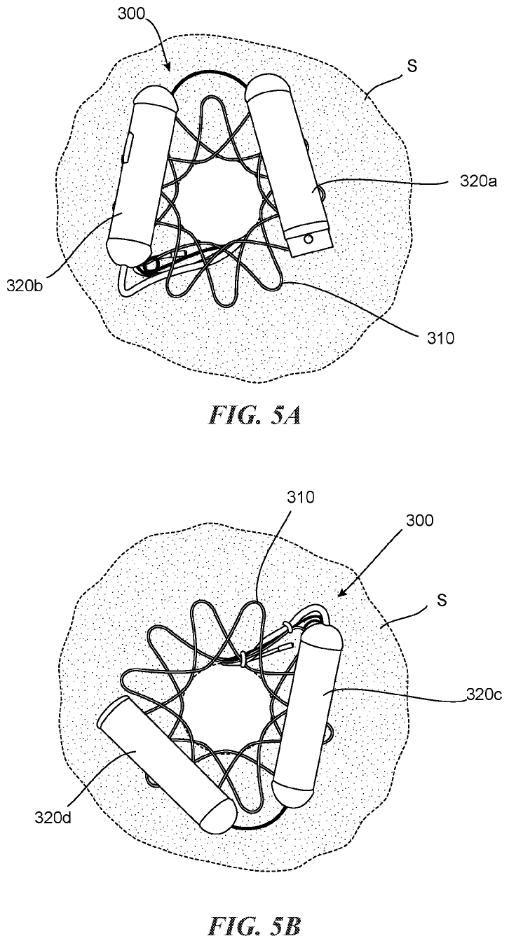

FIGS. 5A and 5B illustrate the system 300 of FIGS. 3A-3C deployed across a septal wall S in accordance with embodiments of the present technology. In particular, FIG. 5A illustrates the system 300 from the left atrium side of the septal wall S, and FIG. 5B illustrates the system 300 from the right atrium side of the septal wall S. The system 300 can be delivered and deployed using a method generally similar to that described with respect to FIGS. 2B-2G for delivering and deploying the system 100. Once deployed, the system 300 can shunt blood between the left atrium and the right atrium via an opening or lumen 316 in the anchoring feature 310.

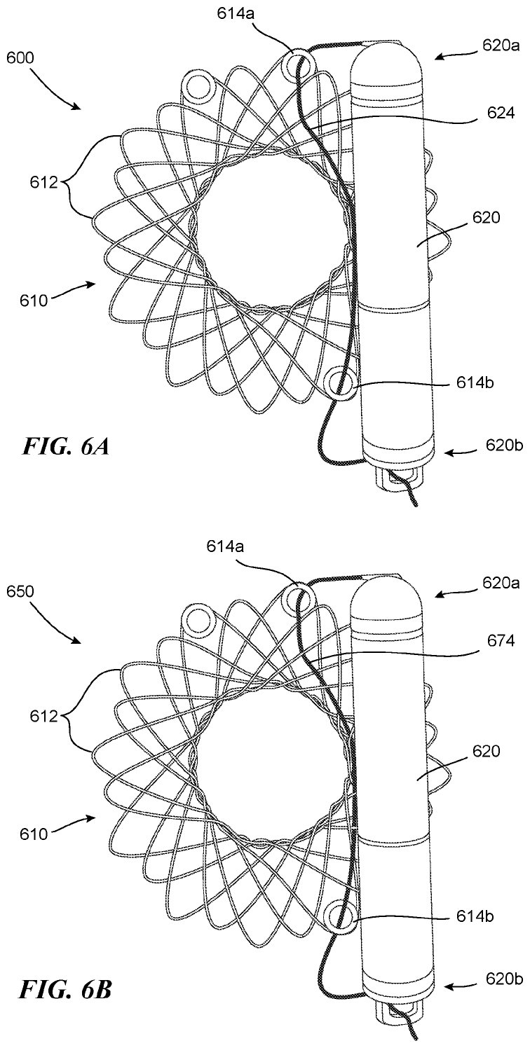

FIG. 6A illustrates an adjustable shunting system 600 in a deployed configuration and configured in accordance with another embodiment of the present technology. Similar to the systems 100 and 300 described with respect to FIGS. 1A-5B, the system 600 can be configured to shunt fluid between a first body region and a second body region when implanted in a patient. For example, the system 600 can be an interatrial shunting system configured to be implanted across a septal wall of a patient to shunt blood between the left atrium and the right atrium.

The system 600 can include certain features generally similar to the systems 100 and 300 described with respect to FIGS. 1A-5B. For example, the system 600 can include a canister 620 and an anchoring or stabilizing feature 610 that is the same as or generally similar to the anchoring feature 110 of the system 100. In the deployed configuration (as shown), the canister 620 is positioned in apposition with petals 612 of the anchoring feature 610. Relative the system 100, however, the illustrated system 600 only includes a single canister 620.

Similar to the anchoring features described previously, the anchoring feature 610 may be composed of Nitinol and exhibit self-expanding properties at body temperature such that, after being initially released from a delivery tool (not shown) in an initial or preliminary position, the anchoring feature 610 will transition its shape toward the deployed configuration. In other embodiments, however, the anchoring feature 610 may be composed of different materials and/or have a different configuration.

The system 600 further includes a tethering element 624 extending between a first portion (e.g., a first end 620a) of the canister 620 and a second, different portion (e.g., a second end 620b) of the canister 620. In the illustrated embodiment, for example, the tethering element 624 extends through both a first eyelet or securement feature 614a and a second eyelet or securement feature 614b of the anchoring feature 610 to help mechanically secure the tethering element 624 to the anchoring feature 610. The tethering element 620 can be similar to the tethering elements described above with reference to FIGS. 1A-4 (e.g., tethering elements 124/126/324/325) and composed of the same or similar materials to those described previously.

The tethering element 624 is expected to help assist with deploying the canister 620 into the desired position/orientation when the system 600 is deployed from a catheter. During deployment, for example, the tethering element 624 can slidably move (through eyelets 614a and 614b) relative to the anchoring feature 610 and can at least partially hold the canister 620 in a desired position/orientation relative to the anchoring feature 610, even if deformation forces are applied against the canister 620. As shown in the embodiment of the system 600 illustrated in FIG. 6A (the deployed configuration), the tethering element 624 is configured to lay generally flat against the anchoring feature 610 and the canister 620 lays in apposition with the petals 612 of the anchoring feature 610. When deployed, this mechanical connection between the canister 620 and multiple points along the anchoring feature 610 is expected to provide improved distribution of the loads between the canister 620 and the anchoring feature 610 during operation as compared with arrangements that include only a single point of contact between the anchoring feature 610 and the canister 620.

While the tethering element 624 is shown slidably received through both eyelets 614 in the embodiment shown in FIG. 6A, in other embodiments the tethering element 624 may be secured or coupled (e.g., via suturing, locking mechanism, etc.) to one of the eyelets 614 such that the tethering element 624 is slidably movable with respect to only a single eyelet (and fixed to the other eyelet). In one specific example, the tethering element 624 may be sutured/fixed to the first eyelet 614a, while remaining slidably movable through the second eyelet 614b during delivery/deployment. In other embodiments, however, the tethering element 624 may not be fixably secured to either eyelet 614, or may have another suitable arrangement relative to the anchoring feature 610.

FIG. 6B illustrates an adjustable shunting system 650 in a deployed configuration and configured in accordance with yet another embodiment of the present technology. The system 650 can be generally similar to the system 600 described above with reference to FIG. 6A, except that the system 650 includes a tethering element 674 interlaced with (e.g., woven through) one or more of the petals 112 as the tethering element 674 extends across the anchoring feature 610. The interlaced arrangement between the tethering element 674 and the anchoring feature 610 is expected to improve/enhance recapturability of the system 650 during operation. The arrangement shown in FIG. 6B is further expected to help enhance deployability of the anchoring feature 610 of the system 650 because of the additional points of contact between the petals 612 of the anchoring feature 610 and the tethering element 674 woven therethrough.

While the embodiments shown in FIGS. 6A and 6B only show a single canister 620, it will be appreciated that the systems 600 and 650, respectively, may include a different number of canisters (two, three, four, etc.). Further, the system 600 and/or the system 650 may include one or more additional features described herein with reference to systems 100 and 300 described herein.

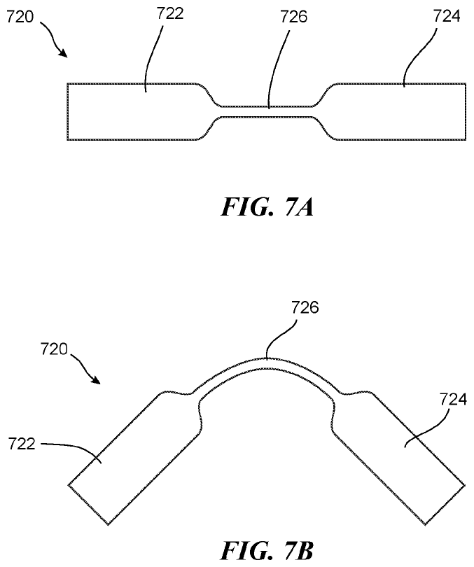

FIGS. 7A and 7B illustrate a canister 720 that can be used with the shunting systems described herein and configured in accordance with select embodiments of the present technology. Referring first to FIG. 7A, the canister 720 can include a first chamber portion 722 and a second chamber portion 724 connected via a narrow central portion 726. In some embodiments, the canister 720 is a single hermetically sealed structure. Similar to the canisters 120, 320 described above, the canister 720 can house one or more energy storage components (e.g., a battery, a capacitor, a supercapacitor, etc.) one or more sensors or associated electronic circuitry (e.g., pressure sensors), an actuator (e.g., a motor), one or more data storage elements, one or more telemetry components, one or more microcontrollers, one or more radios, one or more processors, or the like. Such components are generally located in the first chamber portion 722 or the second chamber portion 724, but in some embodiments may be located partially or fully in the central portion 726. In some embodiments, one or more electrical connections between components housed in the first chamber portion 722 and the second chamber portion 724 may be routed through the central portion 726. In some embodiments, the first chamber portion 722 is configured to reside on a first (e.g., left atrium) side of a tissue structure (e.g., a septal wall), and the second chamber portion 724 is configured to reside on a second (e.g., right atrium) side of the tissue structure. In other embodiments, the first chamber portion 722 and the second chamber portion 724 are configured to reside on the same side of the tissue structure.

The narrow central portion 726 can be configured to undergo a shape change when deployed from a delivery catheter to transition the canister 700 from the delivery configuration shown in FIG. 7A to a deployed configuration, such as that shown in FIG. 7B. For example, the narrow central portion 726 can be at last partially composed of a superelastic material that has been manufactured such that it biases the first chamber portion 722 and/or the second chamber portion 724 toward a desired position (e.g., as shown in FIG. 7B). As another example, the central portion 726 can be composed of a rigid (or at least semi-rigid) and inflexible material that preferentially occupies the deployed configuration shown in FIG. 7B. As yet another example, the central portion 726 can be composed of a malleable material that may be bent into a desired orientation following deployment of the canister 720 from a catheter. Accordingly, the central portion 726 can function similarly to the first tethering element 124 described with respect to FIGS. 1A-2G, and may assist with positioning the canister 720 in and/or retaining the canister 720 at a desired position.

As one of skill in the art will appreciate from the disclosure herein, various components of the systems described above can be omitted without deviating from the scope of the present technology. Likewise, additional components not explicitly described above may be added to the systems without deviating from the scope of the present technology. Moreover, the features described herein can be incorporated into other types of implantable medical devices beyond shunting systems. Accordingly, the present technology is not limited to the configurations expressly identified herein, but rather encompasses variations and alterations of the described systems.

### EXAMPLES

Several aspects of the present technology are set forth in the following examples:

1. A shunting system, comprising:
an anchoring feature configured to extend across a tissue wall separating a first body region and a second body region of a patient;
a first canister housing at least a first electrical component, the first canister being coupled to the anchoring feature and configured to reside within the first body region;
a second canister housing at least a second electrical component, the second canister being coupled to the anchoring feature and configured to reside within the second body region; and
a tethering element extending between the first canister and the second canister, wherein the tethering element is configured to (a) orient the first canister and the second canister in a predetermined configuration when the system is deployed from a catheter, and (b) retain the first canister and the second canister in the predetermined configuration following deployment of the system.

2. The shunting system of example 1 wherein the tethering element is composed at least partially of a material that exhibits elastic properties at body temperature.

3. The shunting system of example 1 or example 2 wherein the tethering element is composed at least partially of Nitinol having an austenite finish temperature of less than body temperature.

4. The shunting system of any one of examples 1-3 wherein the tethering element includes a first loop portion adjacent the first canister and a second loop portion adjacent the second canister, wherein the first and second loop portions bias the first canister and the second canister toward the predetermined configuration.

5. The shunting system of any one of examples 1-4 wherein the tethering element biases the first canister and the second canister into apposition with the anchoring feature.

6 The shunting system of any one of examples 1-4 wherein the tethering element biases the first canister and the second canister into a position that substantially overlaps with the anchoring feature.

7. The shunting system of any one of examples 1-6, further comprising an electrical connection extending between the first canister and the second canister.

8. The shunting system of example 7 wherein the electrical connection is separate from the tethering element.

9. The shunting system of example 8 wherein the electrical connection is sutured to the tethering element along a central portion and un-coupled to the tethering element adjacent the first canister and the second canister.

10. The shunting system of any one of examples 7-9 wherein the electrical connection comprises a conductive wire.

11. The shunting system of claim 1 wherein the first electrical component and/or the second electrical component includes one or more energy storage components, one or more sensors or associated electronic circuitry, an actuator, one or more data storage elements, one or more telemetry components, one or more microcontrollers, one or more radios, and/or one or more processors.

12. The shunting system of any one of examples 1-11 wherein the anchoring feature includes a plurality of first petals, a plurality of second petals, and a central portion extending between the first petals and the second petals, wherein the first petals are configured to be positioned in the first body region and the second petals are configured to be positioned in the second body region to secure the tissue wall therebetween.

13. The shunting system of any one of examples 1-12, further comprising a delivery catheter, wherein the anchoring feature, the first canister, and the second canister are positioned within the catheter in an end-to-end configuration such that they do not overlap.

14. The shunting system of example 13, further comprising:
a first temporary tether extending through the catheter and having a distal end coupled to the first canister and a proximal end accessible external to the patient; and
a second temporary tether extending through the catheter and having a distal end coupled to the second canister and a proximal end accessible external to the patient,
wherein, during a procedure, the distal ends of the first and second temporary tethers are positioned to be manipulated by a clinician external to the patient to assist with positioning of the first and second canisters, respectively.

15. The shunting system of any one of examples 1-14, further comprising one or more mating elements configured to lock the first canister and/or the second canister in the predetermined configuration.

16. The shunting system of example 15 wherein the first canister includes a first mating element and the anchoring feature includes a second mating element, and wherein the first mating element is configured to mate with the second mating element to lock the first canister in the predetermined configuration.

17. The shunting system of any one of examples 1-16 wherein the tissue wall is a septal wall, the first body region is a left atrium of a heart, and the second body region is a right atrium of a heart.

18. A method of deploying a shunting system across a tissue wall dividing a first body region and a second body region of a patient, the method comprising:
advancing a distal end of a catheter carrying the shunting system through the tissue wall and into the first body region, the shunting system including at least a first canister, a second canister, and an anchoring feature,
wherein the first canister, the second canister, and the anchoring feature are positioned in the catheter in an end-to-end arrangement such that the first canister, the second canister, and the anchoring feature do not overlap; and
deploying the shunting system from the catheter, wherein deploying the shunting system includes:
   pushing at least the first canister out of the distal end of the catheter such that the first canister is positioned within the first body region,
   pushing the anchoring feature out of the distal end of the catheter such that the anchoring feature is positioned across the tissue wall, and
   pushing at least the second canister out of the distal end of the catheter such that the second catheter is position within the second body region.

19. The method of example 18, further comprising orienting the first canister and the second canister in a predetermined configuration.

20. The method of example 18 or example 19 wherein the shunting system includes a tethering element coupling the first canister and the second canister, and wherein the tethering element is configured to orient the first canister and the second canister in a predetermined configuration once the system is deployed from the catheter without additional input or manipulation by a clinician.

21. The method of example 20 wherein the tethering element is composed at least partially of a material that exhibits elastic properties at body temperature.

22. The method of example 20 or example 21 wherein the tethering element is composed at least partially of Nitinol having an austenite finish temperature of less than body temperature.

23. The method of any one of examples 20-22 wherein the tethering element includes a first loop portion adjacent the first canister and a second loop portion adjacent the second canister, and wherein the first and second loop portions bias the first canister and the second canister, respectively, toward the predetermined configuration.

24. The method of any one of examples claim 18-23 wherein the shunting system further comprises (a) a first temporary tether extending through the catheter and having a distal end coupled to the first canister and a proximal end accessible external to the patient, and (b) a second temporary tether extending through the catheter and having a distal end coupled to the second canister and a proximal end accessible external to the patient, and wherein:
orienting the first canister and the second canister further comprises manipulating the distal ends of the first temporary tether and/or the second temporary tether external to the patient to assist with orienting the first and second canisters, respectively, to a predetermined configuration.

25. An implantable medical device, comprising:
an anchoring feature configured to extend across a tissue wall separating a first body region of a patient and a second body region of the patient;
a canister configured to carry at least one component of the medical device fluidly sealed therein, wherein, during operation, the canister is configured to be retained by the anchoring feature and positioned to reside within the first body region; and
a tethering element extending between a first portion of the canister and a second, different portion of the canister, wherein the tethering element is configured to (a) orient the canister in a predetermined configuration when the device is deployed from a catheter, and (b) retain the canister in the predetermined configuration following deployment of the device.

26. The implantable medical device of example 25 wherein the tethering element is composed at least partially of a material that exhibits elastic properties at body temperature.

27. The implantable medical device of example 25 or example 26 wherein the tethering element is composed at least partially of Nitinol having an austenite finish temperature of less than body temperature.

28. The implantable medical device of any one of examples 25-27 wherein the anchoring feature includes a plurality of petals, and wherein the tethering feature is configured to be interlaced with one or more of the petals as the tethering element extends between the first and second portions of the canister.

29. The implantable medical device of any one of examples 25-28 wherein the tissue wall is a septal wall, the first body region is a left atrium of a heart, and the second body region is a right atrium of a heart.

### CONCLUSION

Embodiments of the present disclosure may include some or all of the following components: a battery, supercapacitor, or other suitable power source: a microcontroller, FPGA, ASIC, or other programmable component or system capable of storing and executing software and/or firmware that drives operation of an implant: memory such as RAM or ROM to store data and/or software/firmware associated with an implant and/or its operation: wireless communication hardware such as an antenna system configured to transmit via Bluetooth, WiFi, or other protocols known in the art: energy harvesting means, for example a coil or antenna which is capable of receiving and/or reading an externally-provided signal which may be used to power the device, charge a battery, initiate a reading from a sensor, or for other purposes. Embodiments may also include one or more sensors, such as pressure sensors, impedance sensors, accelerometers, force/strain sensors, temperature sensors, flow sensors, optical sensors, cameras, microphones or other acoustic sensors, ultrasonic sensors, ECG or other cardiac rhythm sensors, SpO2 and other sensors adapted to measure tissue and/or blood gas levels, blood volume sensors, and other sensors known to those who are skilled in the art. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. For example, although this disclosure has been written to describe devices that are generally described as being used to create a path of fluid communication between the left atrium and the right atrium, it should be appreciated that similar embodiments could be utilized for shunts between other chambers of the heart or for shunts in other regions of the body.

Unless the context clearly requires otherwise, throughout the description and the examples, the words “comprise,” “comprising,” and the like are to be construed in an inclusive sense, as opposed to an exclusive or exhaustive sense: that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1-17. (canceled)

18. A method of deploying a shunting system across a tissue wall dividing a first body region and a second body region of a patient, the method comprising:
advancing a distal end of a catheter carrying the shunting system through the tissue wall and into the first body region, the shunting system including at least a first canister, a second canister, and an anchoring feature, wherein the first canister, the second canister, and the anchoring feature are positioned in the catheter in an end-to-end arrangement such that the first canister, the second canister, and the anchoring feature do not overlap; and
deploying the shunting system from the catheter, wherein deploying the shunting system includes:
pushing at least the first canister out of the distal end of the catheter such that the first canister is positioned within the first body region,
pushing the anchoring feature out of the distal end of the catheter such that the anchoring feature is positioned across the tissue wall, and
pushing at least the second canister out of the distal end of the catheter such that the second catheter is positioned within the second body region.

19. The method of claim 18, further comprising orienting the first canister and the second canister in a predetermined configuration after deploying the shunting system from the catheter.

20. The method of claim 18 wherein the shunting system includes a tethering element extending between the first canister and the second canister, and wherein the tethering element is configured to orient the first canister and the second canister in a predetermined configuration once the system is deployed from the catheter without additional input or manipulation by a clinician.

21. The method of claim 20 wherein the tethering element is composed at least partially of a material that exhibits elastic properties at body temperature.

22. The method of claim 20 wherein the tethering element is composed at least partially of Nitinol having an austenite finish temperature of less than body temperature.

23. The method of claim 20 wherein the tethering element includes a first loop portion adjacent the first canister and a second loop portion adjacent the second canister, and wherein the first and second loop portions bias the first canister and the second canister, respectively, toward the predetermined configuration.

24-29. (canceled)

30. The method of claim 18 wherein the catheter is a 28 French catheter or smaller.

31. The method of claim 18 wherein the catheter is a 24 French catheter or smaller.

32. The method of claim 18 wherein, when the anchoring feature, the first canister, and the second canister are positioned within the catheter, the anchoring feature is positioned between the first canister and the second canister.

33. The method of claim 18 wherein, when the anchoring feature, the first canister, and the second canister are positioned within the catheter, the anchoring feature, the first canister, and the second canister have an overall axial end-to-end length of between about 20 mm and about 80 mm.

34. A shunting system, comprising:
an anchoring feature transitionable between a collapsed delivery configuration and an expanded deployed configuration, wherein in the expanded deployed configuration the anchoring features is configured to extend across a tissue wall separating a first body region and a second body region of a patient;
a first canister configured to reside within the first body region and housing at least a first electrical component, the first canister being coupled to the anchoring feature such that the first canister has a first predetermined orientation relative to the anchoring feature when the anchoring feature is in the expanded deployed configuration; and
a second canister configured to reside in the second body region on an opposite side of the tissue wall as the first body region and housing at least a second electrical component, the second canister being coupled to the anchoring feature such that the second canister has a second predetermined orientation relative to the anchoring feature when the anchoring feature is in the expanded deployed configuration;
wherein, when the anchoring feature is in the collapsed delivery configuration, the anchoring feature, the first canister, and the second canister are positionable in a delivery catheter in an end-to-end arrangement.

35. The shunting system of claim 34, further comprising the delivery catheter.

36. The shunting system of claim 35 wherein the delivery catheter is a 28 French catheter or smaller.

37. The shunting system of claim 35 wherein the delivery catheter is a 24 French catheter or smaller.

38. The shunting system of claim 35 wherein, when the anchoring feature, the first canister, and the second canister are positioned within the delivery catheter, the anchoring feature is positioned between the first canister and the second canister.

39. The shunting system of claim 35 wherein, when the anchoring feature, the first canister, and the second canister are positioned within the delivery catheter, the anchoring feature does not overlap the first canister and the second canister.

40. The shunting system of claim 35 wherein, when the anchoring feature, the first canister, and the second canister are positioned within the delivery catheter, the anchoring feature, the first canister, and the second canister have an overall axial end-to-end length of between about 20 mm and about 80 mm.

41. A shunting system, comprising:
an anchoring feature transitionable between a collapsed delivery configuration and an expanded deployed configuration, wherein, in the expanded deployed configuration, the anchoring features is configured to extend across a tissue wall separating a first body region and a second body region of a patient;
a first canister configured to reside within the first body region of the patient, the first canister housing at least a first electrical component, wherein the first canister is configured to assume a first predetermined orientation relative to the anchoring feature when the anchoring feature is in the expanded deployed configuration; and
a second canister configured to reside in the second body region of the patient on an opposite side of the tissue wall from the first body region, the second canister housing at least a second electrical component, wherein the second canister is configured to assume a second predetermined orientation relative to the anchoring feature when the anchoring feature is in the expanded deployed configuration;
wherein, when the anchoring feature is in the collapsed delivery configuration, the anchoring feature, the first canister, and the second canister are positionable in a delivery catheter in an end-to-end arrangement.

42. The shunting system of claim 41, further comprising a tethering element extending between the first canister and the second canister, wherein the tethering element is configured to orient the first canister in the first predetermined orientation and orient the second canister in the second predetermined orientation when the anchoring feature is in the expanded deployed configuration.

43. The shunting system of claim 42 wherein the tethering element is composed at least partially of a material that exhibits elastic properties at body temperature.

44. The shunting system of claim 42 wherein the tethering element is composed at least partially of Nitinol having an austenite finish temperature of less than body temperature.

45. The shunting system of claim 42 wherein the tethering element includes a first loop portion adjacent the first canister and a second loop portion adjacent the second canister, and wherein the first and second loop portions bias the first canister and the second canister, respectively, toward the predetermined configuration.

46. The shunting system of claim 41, further comprising an electrical connection extending between the first canister and the second canister.

47. The shunting system of claim 46 wherein the electrical connection is separate from the tethering element.
